(19) United States
(12) Patent Application Publication
(10) Pub. No.: US 2007/0104772 A1
(43) Pub. Date: May 10, 2007

(54) Amorolfine patch for the treatment of onychomycosis

(76) Inventors: Leslie Zanutto, Paulhaguet (FR); Laurent Fredon, Roquefort Les Pins (FR); Claire Mallard, Mougins Le Haut (FR)

Correspondence Address: BUCHANAN, INGERSOLL & ROONEY PC, POST OFFICE BOX 1404, ALEXANDRIA, VA, 22313-1404, US

(21) Appl. No.: 11/527,545

(22) Filed: Sep. 27, 2006

Related U.S. Application Data
(63) Continuation of application No. PCT/FR05/00704, filed on Mar. 23, 2005.

(30) Foreign Application Priority Data
Mar. 29, 2004 (FR) 04/03206

(51) Int. Cl. A61K 9/70

(52) U.S. Cl. 424/449

(57) ABSTRACT

A transungual patch/device useful for the treatment of onychomycosis includes: (i) a backing medium; and (ii) an adhesive matrix layer deposited thereon which contains (a) a thus effective amount of amorolfine active agent or derivative thereof essentially uniformly distributed therethrough, dissolved or dispersed in the adhesive, and (b) at least one absorption promoter for enhancing the absorption of the amorolfine or derivative into the nail onto which the patch/device is applied, the at least one absorption promoter comprising either exaltolide alone, or at least two such absorption promoters selected from among urea, N-acetylcysteine and lactic acid.

### CROSS-REFERENCE TO PRIORITY/PCT APPLICATIONS

This application claims priority under 35 U.S.C. § 119 of FR 04/03206, filed Mar. 29, 2004, and is a continuation of PCT/FR 2005/000704, filed Mar. 23, 2005 and designating the United States (published in the French language on Oct. 6, 2005 as WO 2005/092299 A1; the title and abstract were also published in English), each hereby expressly incorporated by reference and each assigned to the assignee hereof.

### BACKGROUND OF THE INVENTION

1. Technical Field of the Invention

The present invention relates to a transungual device, patch or bandage containing amorolfine or derivative thereof, in particular its hydrochloride derivative, that is effective in the treatment of onychomycosis.

2. Description of Background and/or Related and/or Prior Art

Onychomycosis is a mycosal infection of the nail, manifested by opaque, white, thick, friable and brittle nails. It generally affects more than one nail. Onychomycosis affects 2 to 13% of the population, increasing to around 15-20% in the age group 40 to 60.

Commonly used treatments for onychomycosis are grouped into three categories:
(1) systemic and local antifungal treatments;
(2) surgical interventions to remove all or part of the infected nail followed by a topical treatment for the exposed tissues; or
(3) topical applications of creams, lotions, gels or solutions on the infected nail.

These various approaches present a number of drawbacks.

Systemic administration (oral route) of antifungal agents for the treatment of onychomycosis only provides a therapeutic effect in the long term. For example, oral treatment with the antifungal compound ketoconazole typically requires the administration of 200 to 400 mg per day for six months before a significant therapeutic benefit is obtained. Moreover, these antifungal agents can have significant undesirable long term side-effects.

Ablation of the nail by surgical means also has a certain number of drawbacks, including in particular pain and discomfort associated with the surgical intervention and the unattractive appearance of the nail.

In the case of the usual topical treatments using creams, lotions, gels or solutions, diffusion of the active agent through the surface of the nail is very limited and the duration of the treatment is particularly long. Moreover, these forms of topical dosage fail to maintain the active agent in contact with the nail for an extended period of time and bandages must therefore be used.

Another known topical form is nail varnish (Murdan et al., International Journal of Pharmaceutics, 236 (2002), 1-26). Loceryl® is an example of a varnish composed of amorolfine (5%), Eudragit RL 100, glycerol triacetate, butyl acetate, ethyl acetate and ethanol. The varnish is applied to the nail plate and dried for several minutes to evaporate the solvents and leave a film of water-impermeable polymer on the surface of the nail. The active agent is then released from the film and diffuses through the nail plate.

Loceryl® is applied once or twice a week for six months to the fingernails and for between nine and twelve months in the case of the toenails. The duration of the treatment essentially depends on the severity and location of the infection and the affected surface area of the nail.

The treatment of nails using a varnish is thus found to be very demanding in that it is repetitive, requires maintenance of the nail before each application, and requires particular care to avoid any contamination of unaffected nails.

WO 99/40955 describes a transdermal and transungual patch for the treatment of onychomycosis including an antifungal agent, such as fluconazole, terbinafine, clotrimazole, miconazole and ketoconazole or their salts, but amorolfine hydrochloride is not indicated.

### SUMMARY OF THE INVENTION

It has now been found that amorolfine, particularly in hydrochloride form, is capable of diffusing effectively through the nail plate by means of a transungual bioadhesive matrix device. Such a transungual device or patch is intended to be applied exclusively on the surface of the infected nail.

The present invention thus features a transungual device, patch or bandage including:
(i) a backing medium; and
(ii) an adhesive matrix layer including a) an active agent selected from amorolfine and its derivatives, dissolved or dispersed in an adhesive and b) either a single absorption promoter, namely, exaltolide, or at least two absorption promoters selected from the group consisting of urea, N-acetylcysteine and lactic acid.

In the following text, unless otherwise indicated, the proportions of the different constituents of the adhesive matrix layer are expressed in percentage by weight (m/m) of the dry mass of the adhesive matrix layer.

### DETAILED DESCRIPTION OF BEST MODE AND SPECIFIC/PREFERRED EMBODIMENTS OF THE INVENTION

Preferably, amorolfine or derivative thereof are present in a quantity from 2 to 70% by weight of the matrix adhesive layer, preferably from 20 to 40% by weight. In a preferred manner, the adhesive is a pressure-sensitive adhesive and is preferably selected from polyacrylates, polyisobutylenes, silicones or a mixture thereof.

The adhesive can represent from 10 to 98% by weight of the adhesive matrix layer and more preferably from 25 to 85% by weight.

In a preferred manner, the backing medium includes a polymer selected from polyurethane, polyethylene, poly(vinyl ethylene-co-acetate), polyvinyl chloride or a mixture thereof.

The transungual device according to the invention is anhydrous.

The term anhydrous is understood to mean a device containing less than 2.5% water.

The quantity of each absorption promoter preferably ranges from 1 to 30% by weight of the adhesive matrix layer, and more preferably from 2.5 to 15% by weight.

In a preferred embodiment, the adhesive matrix layer contains two adsorption promoters selected from among urea, N-acetylcysteine and lactic acid.

In another preferred embodiment, the absorption promoter is urea associated with lactic acid or with N-acetylcysteine.

In a first embodiment, the absorption promoter is a urea/lactic acid mixture, each preferably independently comprising from 2 to 6% by weight of the adhesive matrix layer.

In a second embodiment, the absorption promoter is a urea/N-acetylcysteine mixture, each independently comprising from 2 to 10% by weight of the adhesive matrix layer.

In another embodiment, a single absorption promoter is used and is exaltolide alone, which preferably ranges from 10 to 20% by weight of the adhesive matrix layer.

In another preferred embodiment, the device additionally includes a detachable outer covering which is deposited on the adhesive matrix layer.

In a second aspect, this invention features a method of preparing a device according to the invention including the steps of:
i) preparing a mixture including (1) adhesive, (2) either a single absorption promoter, namely, exaltolide, or at least two absorption promoters selected from the group consisting of urea, N-acetylcysteine and lactic acid, and (3) an active agent selected from amorolfine and its derivatives, in a solvent, said adhesive being miscible with said solvent;
ii) depositing the mixture obtained onto the backing medium; and
iii) evaporating the solvent.

In a preferred embodiment, the adhesive is a polyacrylate. In this case, the solvent is preferably ethanol.

In another preferred embodiment, the adhesive is a silicone. In this case, the solvent is preferably dichloromethane.

In one embodiment, the amorolfine or its derivatives are dissolved in the mixture obtained at i).

The expression dissolved form is understood to mean a molecular dispersion in a liquid, no crystallization of the active agent being visible to the naked eye or even under an optical microscope with cross polarization.

In another embodiment, the amorolfine or its derivatives are dispersed in the mixture obtained at i).

Preferably, the mixture obtained at i) includes two promoters to enhance absorption of the amorolfine into the nail. Alternatively, the mixture obtained at i) preferably includes exaltolide as the sole absorption promoter of the amorolfine into the nail. In a particular embodiment, the mixture obtained at i) additionally includes a co-solvent capable of dissolving amorolfine or its derivatives and/or the adsorption promoter(s) in the mixture.

The co-solvent is preferably isopropanol.

The solvent and the co-solvent, if any, can represent from 2 to 90% by weight of the mixture obtained at step i).

In a preferred manner, the method includes a step iv) involving placement of the detachable outer covering.

In a third aspect, the present invention features the use of amorolfine or its derivatives to manufacture a medicament intended for the treatment of onychomycosis, said medicament being in the form of a transungual device, patch or bandage according to the invention.

In a fourth aspect, this invention features the use of amorolfine or its derivatives to manufacture a medicament intended for the treatment of onychomycosis, said medicament being in the form of a transungual device, patch or bandage obtained by the method according to the invention.

In a first aspect, the present invention features a transungual device, patch or bandage including:
(i) a backing medium; and
(ii) an adhesive matrix layer including a) amorolfine or one of its derivatives, dissolved or dispersed in an adhesive and b) either a single absorption promoter, namely, exaltolide, or at least two absorption promoters selected from the group consisting of urea, N-acetylcysteine and lactic acid.

The term amorolfine is understood to mean base amorolfine. The expression amorolfine derivatives is understood to mean in particular its pharmaceutically acceptable salts, and more particularly its hydrochloride derivative, hereinafter designated amorolfine HCl.

The expression pharmaceutically acceptable salts is understood to mean salts compatible with the skin, the mucous membranes and/or other external parts of the body.

Amorolfine HCl or amorolfine hydrochloride is a hydrochloride derivative of amorolfine and designates the hydrochloride of the cis-4-[3-[4-(1,1-dimethyl-propyl)phenyl]-2-methylpropyl]-2,6-dimethyl-morpholine.

This chemical compound exerts a fungistatic and fungicidal action by inhibiting the synthesis of sterols in the cell membrane of fungi such as yeasts, dermatophytes, moulds and dematiaceous (black) fungi.

The expression transungual device, patch or bandage is understood to mean a rigid or flexible element intended to be applied exclusively on the nail and to adhere thereto, allowing the release and penetration of an active agent on a target area of the nail. This device is distinguished from the traditional transdermal device in that in no case does it project beyond the nail surface; it does not cover the adjacent parts of the skin, thus eliminating the risks of systemic transfer.

For the sake of simplicity, the expressions “transungual device” and “patch” or “bandage” will be used interchangeably.

The patches of the invention can have various structures.

By way of illustration, a single-layer matrix device and a multi-layer matrix device are shown respectively in FIG. 1 and FIG. 2.

The single-layer matrix device comprises a backing medium (1), a single adhesive matrix layer (2) and a detachable outer covering (3).

The multi-layer matrix device comprises at least two matrix layers, at least one of the two layers being an adhesive layer. Each is separated by a membrane (4) which allows gradual release of the amorolfine or one of its derivatives, optionally in combination with other active agents.

Backing Medium Layer

The backing medium of the transungual device/patch is the part of the patch that is visible when it is applied on the skin.

It serves to maintain the active agent in contact with the nail to increase the diffusion of the active agent inside the nail.

It may be occlusive or non-occlusive.

The backing medium also serves to protect the layer in contact with the nail against any contamination.

For reasons of cosmetic appearance and discretion, the backing medium will preferably be colorless or colored.

It will preferably have sufficient flexibility to effectively conform to the surface of the nail even when the surface is irregular.

It is composed of polymers selected principally so as to present an inertia and a chemical resistance vis-a-vis the components of the patch formulation, i.e., in particular amorolfine or one of its derivatives, the adhesive and any solvents, as well as the absorption promoter(s).

The selection of backing medium may additionally depend on the desired appearance, the requirement or otherwise for occlusion, and the need or otherwise for flexibility. To this end, the stretch resistance and permeability to water vapor and oxygen of the polymers may be taken into consideration.

Preferably, the backing medium includes a polymer selected from among polyurethane, polyethylene, poly(vinyl ethylene-co-acetate), polyvinyl chloride or a mixture thereof.

As examples of backing medium suitable for the manufacture of a patch according to the invention, representative are the compounds marketed under the trademarks Dow BLF 2015, Dow BLF 2023, Dow BLF 2050, Dow BLF 2052, Dow BLF 2057 and Dow BLF 2080 by Dow Chemicals.

Adhesive Matrix Layer

For the purposes of the present invention, the term “matrix” or “adhesive matrix layer” means a mixture of at least one active agent including amorolfine or one of its derivatives homogeneously dispersed or dissolved in a biocompatible and pressure-sensitive adhesive, said mixture additionally containing either exaltolide or at least two promoters to enhance the absorption of the homogeneously dissolved or dispersed amorolfine or one of its derivatives.

In the case of a single-layer device, this matrix layer has a surface directly in contact with the backing medium, and a second surface directly in contact with the detachable outer covering to be removed when the patch is applied on the area to be treated.

During use, this detachable outer covering is removed from said surface of the matrix layer which is then applied directly onto the nail.

The active agent and the absorption promoters diffuse through the adhesive and its mixtures, if any, to the surface of the nail.

This matrix layer therefore constitutes a single dosage form or unit dosage amount of a composition of amorolfine or one of its derivatives in an adhesive medium containing either exaltolide or at least two promoters to enhance the absorption of the active agent into the nail.

Adhesive

For the purposes of this description, the term adhesive denotes a polymer or a mixture of polymers that are chemically inert vis-a-vis the different components of the matrix, including in particular amorolfine or one of its derivatives and the absorption promoters.

Its principal function is to ensure the adhesion of the patch on the nail.

It is advantageously selected so that it leaves no residue on the nail when the patch is removed.

It preferably exhibits good adherence vis-à-vis the backing medium.

Adhesives suitable for use according to the invention include Pressure Sensitive Adhesives (PSA). Among PSA adhesives suitable for the manufacture of a patch according to the invention, the following two main classes are recognized:

Solvent-free adhesives referred to as “hot melt” adhesives. These are characterized by a solid state at temperatures below 180° F. and have low viscosities above 180° F. They can therefore be formulated in the hot condition. Among the solvent-free adhesives, representative are the various copolymers such as in particular ethylene vinyl acetate copolymers, styrene-isopropene-styrene copolymers, styrene-butadiene-styrene copolymers, reactive polyurethanes (PUR). Generally, these polymers do not in themselves have the performance characteristics required for the manufacture of a finished product. Therefore, this type of “hot melt” solvent-free adhesive will preferably be used in the presence of waxes, antioxidants, plasticizers or other additives the purpose of which will be to improve the performance of the adhesive.

Adhesives containing solvent, referred to as solvent-based adhesives. These adhesives in solution have good adhesion properties when used by themselves and do not therefore require plasticizers or other additives. Furthermore, these compounds can be formulated in the cold condition.

Therefore, in view of their properties as defined above, solvent-based adhesives are the preferred adhesives to be used to fabricate a patch according to the invention.

Among the solvent-based adhesives suitable for use to fabricate a patch according to the invention, those selected from polyacrylates, polyisobutylenes, silicones or a mixture thereof are particularly preferred.

The term “polyacrylate” or “acrylic” is understood to mean a polymer having the formula: 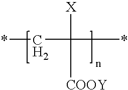
in which

X preferably is an atom of hydrogen or a C₁-C₁₂ alkyl radical;

Y preferably is an atom of hydrogen or a C₁-C₁₂ alkyl radical optionally substituted;

n denotes the number of monomer units.

The term “polyisobutylene” is preferably understood to mean a polymer having the formula: 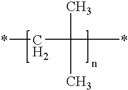
in which n denotes the number of monomer units.

The term “silicone” is understood to mean a polysiloxane polymer having the formula: (RR′SiO)n, in which R and R′ are identical or different and preferably represent an atom of hydrogen or a C₁-C₁₂ alkyl radical.

The term “alkyl” describes an aliphatic hydrocarbon radical which can be linear or branched having 1 to 12 carbon atoms, preferably 1 to 6 carbon atoms in the chain. Branched means that one or more lower alkyl groups such as methyl, ethyl or propyl are attached to a linear alkyl chain.

The expression “lower alkyl” means 1 to 4 carbon atoms in the chain which can be linear or branched.

The alkyl can be substituted by one or more “alkyl group substituents” which may be identical or different and include the hydroxyl, amino, alkylamino or dialkylamino groups.

By way of examples of acrylic adhesives, mention may be made in particular of the vinyl acetate acrylic polymers marketed in solution form by National Starch, under the trademark Duro-Tak® 387-2287, Duro-Tak® 387-2516 and the acrylic polymers marketed in solution form under the trademark Duro-Tak® 387-2353.

By way of examples of polyisobutylenes, mention may be made in particular of paraffinic hydrocarbon polymer type compounds composed of macromolecules with long linear chain terminating in olefinic bonds. These compounds are marketed under the trademark Vistanex® LM MS LL and Vistanex® MM L80, which are presented in solid form.

By way of example of silicones, mention may be made in particular of pressure sensitive adhesives such as tetra (trimethylsiloxy) silane and in particular the compounds marketed by Dow Corning under the trademark BIO PSA®, particularly the BIO PSA 4200® series, and more particularly the adhesives BIO PSA 7-4102®, BIO PSA 7-4202®, and BIO PSA 7-4302®. These three compounds are presented in solution form and differ by their adhesive properties.

Within the matrix layer, the adhesive is present in a quantity preferably ranging from 10 to 98% by weight of the adhesive matrix layer and more preferably from 25 to 85% by weight.

Amorolfine or Its Derivatives

Amorolfine or one of its derivatives may be in the dissolved state or in the dispersed state in the adhesive matrix layer.

In all cases, it is desirable that the amorolfine or one of its derivatives is uniformly distributed within the adhesive matrix layer.

More preferably, it will have a uniform particle size distribution.

Preferably, amorolfine or one of its derivatives is present in the adhesive matrix layer in a quantity from 2 to 70% by weight of the adhesive matrix layer and more preferably from 20 to 40% by weight of the adhesive matrix layer.

Preferably, amorolfine HCl is included in the compositions according to the present invention.

Amorolfine or one of its derivatives can be present in combination with another active substance suited for transungual application having biological or pharmacological properties, in particular antibiotic or antifungal, by topical application on the area of the nail to be treated.

Nail Absorption Promoters

The adhesive matrix layer additionally includes either exaltolide or at least two absorption promoters to enhance the absorption of amorolfine or its derivatives into the nail.

The expression “nail absorption promoter” is understood to mean pharmaceutically acceptable chemical compounds capable of increasing the permeability of a biological membrane such as the skin or the nail vis-à-vis amorolfine or its derivatives, thereby increasing the penetration kinetics of amorolfine or its derivatives through the membrane.

This penetration kinetics can be measured using a cellular diffusion apparatus such as Franz cells described by Merrit et al. (Diffusion Apparatus for Skin Penetration, J. Controlled Release, 1984, 1, 161-162).

Absorption promoters are well known in the current art and can include in particular α-hydroxyacids, fatty acid esters and amides thereof, fatty alcohols, fatty acids and esters of glycerol in particular 2-(2-ethoxyethoxy)-ethanol, glycerolmonolaurate, propylene glycol, polyethylene glycols, polyglycosylic glycerides, unsaturated polyglycols (Labrafil M1 944CS®, Gattefosse), saturated polyglycerides (Labrasol, Gattefosse), Labrafac HydroWL1219® (Gattefosse), decylmethylsulfoxide, pyrrolidones, salicyilic acid, lactic acid, isopropyl myristate, dimethylformamide, dimethylacetamide, sodium dodecylsulfate, phospholipids, Transcutol® (Gattefosse), mixtures of oleic acid and 2-(2-ethoxyethoxy)-ethanol, oleic acid and Labrafil®, these oleic acid mixtures preferably being in a ratio of approximately 1:1. Enzymatic compounds, such as proteolytic enzymes which facilitate penetration of the active agents through the keratinous tissues or through the nail, can also be used as absorption promoters. By way of non-limitative examples of fatty acids suitable for use according to the invention, mention may be made of the capric, lauric, myristic, palmitic, stearic, arachidic, behenic, lignoceric, myristoleic, palmitoleic, petroselinic, oleic, linoleic and linolenic acids.

Other known absorption promoters work by virtue of hydrolysis, keratolysis, denaturation or other equivalent mechanism which destroy the nail or the membrane. By way of example of absorption promoters that work in this manner, mention may be made of urea, amino acids including sulfydryl groups, alkylsulfoxides, and any equivalent compound which works by destroying or denaturing the nail and/or the membrane thereby enabling the pharmaceutical compound to penetrate the deeper layers of the membrane.

By way of example of absorption promoters, mention may be made in particular of urea, exaltolide, N-acetylcysteine and lactic acid or a mixture thereof, urea combined with lactic acid or with N-acetylcysteine, and exaltolide alone, being particularly preferred.

Preferably, the absorption promoters are dissolved uniformly in the adhesive matrix layer.

The quantity of each absorption promoter is from 0.1 to 30% by weight of the adhesive matrix layer, more preferably from 1 to 20% by weight and even more preferably from 2 to 10% by weight.

More specifically, when the absorption promoter is urea in combination with lactic acid, it is particularly preferred that the urea and the lactic acid each independently represent 2 to 6% by weight of the adhesive matrix layer.

When the absorption promoter is urea in combination with N-acetylcysteine, it is particularly preferred that the urea and the N-acetylcysteine each independently represent from 2 to 10% by weight of the adhesive matrix layer, and more preferably that they are present at the rate of 5 to 10% in the case of urea and 2 to 6% in the case of N-acetylcysteine by weight of the adhesive matrix layer.

Additives

The adhesive matrix layer contained in the transungual device/patch suitable for use according to the invention is characterized by the fact that it does not include any additive, the latter having been eliminated by evaporation.

In effect, during the manufacture of the transungual device according to the invention, the use of organic or aqueous solvents and/or co-solvents is necessary for homogenization of the mixture including the adhesive, at least two adsorption promoters and amorolfine or one of its derivatives. Such solvents and/or co-solvents are then eliminated from the adhesive matrix layer by evaporation.

Such solvents and/or co-solvents are the only additives suitable for use according to the present invention. They are present in the adhesive matrix layer only during its manufacture.

Detachable Outer Covering

The transungual device generally includes a detachable outer covering.

The expression detachable outer covering is understood to mean a protective medium in direct contact with the adhesive matrix layer that is removed just before application on the skin. It must therefore be chemically inert and impermeable vis-à-vis the patch formula.

By way of example of material suitable for use as the detachable outer covering, mention may be made in particular of aluminum sheet, paper incorporating a sheet of polyethylene or a silicone polyester.

Separating Membranes

The patch may additionally include one or more separating membranes or layers: this then constitutes in particular a multi-layered matrix device such as depicted in FIG. 2.

These separating membranes are formed from chemically inert polymers such as polyethylene or polypropylene.

These membranes can be porous or non-porous.

In the case of porous membranes, the amorolfine or its derivatives are released by direct diffusion through the membrane.

In the case of non-porous membranes, the rate of passage of the amorolfine or its derivatives depends on its solubility in the membrane and the thickness of the latter.

The patches according to the invention can be prepared by conventional techniques such as coating by the so-called “hot melt” method, i.e., in the absence of a solvent, or “solvent phase” method.

In a second aspect, the present invention features a method of preparing a transungual device including the steps of:
i) preparing a mixture including (1) adhesive, (2) either exaltolide or at least two absorption promoters selected from the group formed by lactic acid, N-acetylcysteine and urea, and (3) amorolfine or one of its derivatives, in a solvent, said adhesive being miscible with said solvent;
ii) laying the mixture obtained onto the backing medium; and
iii) evaporating the solvent.

This process is based on the principle of the conventional technique of solvent phase coating.

Step (i) Preparation of the Adhesive Mixture

The choice of solvent is essentially determined by the nature of the adhesive.

It is selected in relation to its ability to dissolve the adhesive and its volatility.

In effect, too high a volatility makes it difficult to obtain uniform bedding.

To this end, the solvent can, for example, be selected from among carboxylic esters, halogenated hydrocarbons, aliphatic hydrocarbons, ethers, ketones, non-protic polar solvents and alcohols, in particular alcanols.

As examples of carboxylic esters, mention may be made in particular of methyl acetate, ethyl acetate, isopropyl acetate, butyl acetate, amyl acetate, and ethyl propionate.

Examples of halogenated hydrocarbons are in particular chloroform, dichloromethane, and 1,2-dichloroethane.

Examples of aliphatic hydrocarbons include in particular pentane, hexane, heptane, and octane.

Examples of ethers are in particular diethyl ether, dibutyl ether, dioxane, and tetrahydrofuran.

As examples of ketones, mention may be made of acetone and methylethylketone.

As examples of non-protic polar solvents, mention may be made of acetonitrile, N,N-dimethylformamide, and dimethylsulfoxide.

In a preferred embodiment, the adhesive used at step i) is a polyacrylate. In this case, alcohols are preferred as the solvent, and more preferably ethanol.

In another preferred embodiment, the adhesive used at step i) is a silicone. In this case, halogenated hydrocarbons are preferred as the solvent, such as dichloromethane in particular.

The choice of solvent can also depend on the state in which it is desired to obtain the amorolfine or its derivatives, i.e., dissolved or dispersed.

Thus, as the case may be, where several solvents are possible, this solvent can be selected to also dissolve the other constituents of the matrix layer such as the amorolfine or its derivatives and/or the absorption promoter(s).

However, the use of a co-solvent or a mixture of co-solvents may prove necessary in some cases.

The choice of co-solvent depends on the nature of the adhesive solution and on the nature of the constituent that it is wished to dissolve.

The co-solvent must be compatible with the adhesive solution, and must in particular be miscible vis-à-vis the solvent and the adhesive, and optionally with the other constituents.

It can in particular be selected from halogenated hydrocarbons such as dichloromethane, alcohols such as ethanol or isopropanol or a mixture thereof.

In particular, given that amorolfine HCl is particularly soluble in ethanol, the latter can be effective as a co-solvent with a solvent other than ethanol.

In the case of acrylic adhesives, ethanol can therefore advantageously be used to dissolve both the adhesive and the amorolfine HCl without it being necessary to add a co-solvent to dissolve the latter.

By way of example, ethanol or isopropanol, alone or in conjunction with dichloromethane, are particularly effective as co-solvents to dissolve the absorption promoter urea/lactic acid combination, in particular with silicone adhesive. Isopropanol is particularly preferred for this purpose.

Of course, one skilled in the art may, in light of his/her general knowledge, determine which co-solvents are most appropriate to the conditions at hand and to the desired dissolved or dispersed state of the constituents.

The combination of solvents and co-solvent will be present in proportions ranging from 2 to 90% by weight, preferably from 10 to 75% by weight of the mixture obtained at step (i) and more preferably from 20 to 70%.

Step (ii) Deposition

The more or less viscous solution or suspension obtained at step i) is coated onto the backing medium.

This step can be accomplished using a commercially available film applicator.

Step (iii) Evaporating the Solvent

The solvent is evaporated either in the ambient air, or by applying heat using a heating base or an oven for example.

In a third aspect, this invention features the use of amorolfine or one of its derivatives to manufacture a medicament designed for the treatment of onychomycosis, said medicament being in the form of a transungual device/patch described above.

In a fourth aspect, this invention features the use of amorolfine or one of its derivatives to manufacture a medicament designed for the treatment of onychomycosis, said medicament being in the form of a transungual device obtained by the method as defined in the present description.

All types of onychomycosis are targeted, i.e., in particular distal-subungual onychomycosis, proximal-subungual white onychomycosis, superficial white onychomycosis and onychomycosis caused by a Candida strain.

The patch of amorolfine or one of its derivatives according to the invention is a form of administration that offers numerous advantages in the treatment of onychomycoses, in particular in that it facilitates local treatment and is simple and quick to apply.

It also has the advantage of increasing the contact time between the nail and the active agent without the need for manipulations, and of releasing the active agent continuously over an extended period.

The patches according to the present invention also have the advantage of avoiding any step involving pretreatment of the nail before application and in particular pretreatment by abrasion.

In another preferred embodiment according to the invention, the patch can be used in conjunction with a systemic treatment for treatment of the nail. A combination of the patch according to the invention with a systemic treatment has in particular proven effective in the treatment of pathologies of the nail, and more particularly in the treatment of onychomycosis.

The patch according to the invention can be made in any size and shape suitable for the required use, such as circular, oval, rectangular or square shapes. Preferably, the patch according to the invention will have a shape best adapted to the shape of the nail, i.e., of the oval type. The preferred size of the patch according to the invention is from 0.5 cm² to 4 cm² and selected in relation to the location of the area to be treated.

Another advantage of the patch according to the invention is that it can be placed on the nail for a week at a time without having to be changed each day. Thus, an excess of amorolfine or its derivatives is preferably used to ensure that the amount of active agent within the patch will be present in sufficient quantity after a week of application.

### BRIEF DESCRIPTION OF THE DRAWINGS

FIG. 1 is a diagrammatic representation of a single-layer matrix device/patch.

FIG. 2 is a diagrammatic representation of a multi-layer matrix device/patch.

FIG. 3 presents, for different patch formulations, an evaluation of the release of amorolfine HCl, expressed in terms of the mass of inhibition zones.

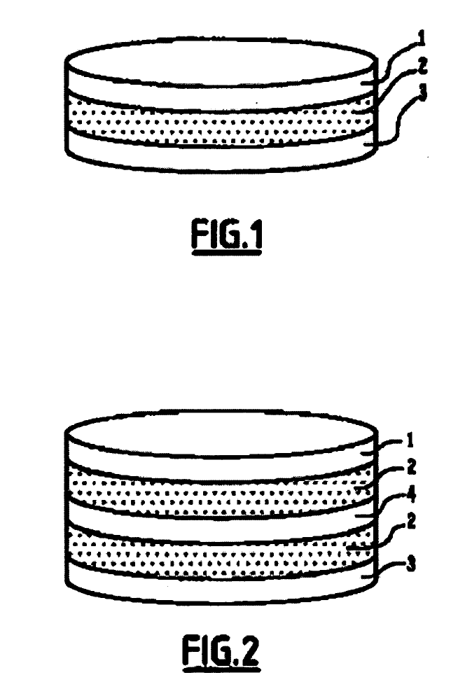

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

Apparatus and Methods

Backing Medium Layers

The backing media used, with the designations Dow BLF 2023® and Dow BLF 2080®, are marketed by Dow Chemical.

Adhesives

The acrylic polymer used is a polymer in solution in ethyl acetate marketed principally under the trademark Duro-Tak® 387-2516 by National Starch.

The silicone polymer BIO PSA 7-4102® is a polymer in solution in ethyl acetate supplied by Dow Corning.

Detachable Outer Covering

The detachable outer covering 3M 9956® used is a polyester film supplied by 3M. The side in contact with the substrate is treated with a fluorinated polymer. It is compatible with the adhesives used. It is transparent, heat sensitive and its thickness is 74±5 μm.

General Procedure for the Preparation of Patches

The patches were prepared by the solvent phase coating technique including:

mixing of all the constituents of the adhesive matrix layer in the solvent, the adhesive being necessarily miscible in this solvent to ensure good coating homogeneity; the mixture is stirred using a magnetic stirrer until a homogeneous preparation is obtained;

about 30 g of the preparation are taken to apply the coating of the more or less viscous solution obtained onto the backing medium;

finally, evaporating the solvent, in the air under an extraction hood for 24 hours at ambient temperature.

The m/m percentages represent the percentages of the various constituents expressed relative to the dry mass of the matrix layer after evaporation of the solvents.

The coating was performed using an Erichsen KCC 101 film applicator with 400 μm or 100 μm thick spiral wire coating bars.

In the tables below, the percentages by weight correspond to the weight of the different constituents of the solution intended to form the adhesive matrix layer after evaporation of the solvents.

Analysis of Patches

The possible formation of amorolfine HCl crystals and the coating uniformity of the patches are observed under a microscope at a magnification ranging from ×50 to ×250 and the images are taken using the “Kappa” image acquisition software.

Microbiological Evaluation Test Procedure

This test serves to determine the efficacy of the formulations made.

The experiment was conducted on pig's hooves which are physiologically similar to human nails.

Principle of Manipulation

The patches are placed on pig's hooves that have first been rehydrated. The hooves are placed on a Sabouraud agar (AES Laboratory reference AEB152352) infected by an inoculum of spores of Trictophyton rubrum, which is implicated in onychomycoses. The agar samples and hooves are then placed in a refrigerator at +4° C. for a determinate period to allow the active agent to diffuse through the hoof then the agar. The agar samples are then placed in an incubator for 48 hours at +32.5° C., which is the ideal temperature for the proliferation of Trichophyton rubrum.

This strain does not grow in the presence of amorolfine HCl. Inhibition zones on the agar can therefore be observed, thereby enabling the release of the amorolfine HCl to be evaluated.

Manipulation Procedure

The Manipulation Is Performed, in Sterile Conditions, in Several Steps:

Rehydration of the pig's hooves, preparation of the infected agars, application of patches on the pig's hooves, refrigeration at +4° C. for a defined period, incubation at +32.5° C. for 48 hours,

Reading of Results

Rehydration of Pig's Hooves:

The pig's hooves are first scraped to remove all of the flesh and are then autoclaved. It is then necessary to rehydrate them before use. A zone delineated by a ring indicates an area on which the formula is deposited. This ring should be manually glued to the hoof before rehydration. These two operations are performed at the same time:

place a sterile gauze in a round Petri dish,

moisten with sterilized water,

cut out the roundels,

place glue on the roundels,

take a hoof, check its integrity,

glue the roundel onto the hoof, making sure that no excess glue appears inside the roundel,

place the hoof on the moist gauze and leave while preparing the culture.

Preparation of Infected Agars

The different steps are as follows:

take up the working cultures in inclined tubes,

scrape these cultures with Sabouraud broth (AES Laboratory reference 111 105) to collect the spores,

filter the suspension to eliminate all residues of agar,

dilute so as to have sufficient suspension to infect the agars (i.e., 1 ml of suspension per 100 ml of agar),

make a 10-fold dilution of the suspension,

place the dilution in a Thoma cell,

leave to rest for a quarter of an hour,

count the spores under the microscope,

calculate the number of colonies in the suspension,

raise this number to the power 7 or 8,

if the concentration is good the manipulation continues.

If not, dilute the initial solution or re-scrape the cultures to collect new spores.

place the solution in a vortex stirrer to ensure good homogeneity,

take the necessary quantity of suspension,

add the suspension to the Sabouraud agar,

stir well,

place the dishes flat using a level and modeling paste, close to a Bunsen burner,

fill the Petri dishes by pouring 250 ml of infected agar into each dish,

leave to set en masse, leaving the dish lids ajar,

close the dishes,

place them under a laminar flow hood,

fully open the dishes,

leave them to dry completely for half an hour.

Application of Patches

The manipulation steps are as follows:

cut out a sample using a die-cutter of precise diameter (8 mm),

stick it onto the hoof inside the roundel,

place the hooves on the agar, with four hooves per dish,

then place the dishes in the refrigerator (+4° C.) for a determinate period.

Diffusion and Incubation

Diffusion of the active agent through the hoof and the agar takes place during the refrigeration period as the trychophyton is unable to grow. Incubation corresponds to the period during which the fungus is able to grow.

The final steps are as follows:

remove the dishes from the refrigerator after the specified time,

remove the hooves using tweezers,

place the dishes in an oven (+32.5° C.) to allow the Trichophyton rubrum to develop; 48 hours is generally a sufficient period in which to obtain clear inhibition zones.

Reading of Results

When the fungus has grown sufficiently, inhibition zones appear indicating the diffusion of active agent through the hoof to the agar. The inhibition zone is larger or smaller depending on the degree of diffusion. The results are determined by comparing these zones. The operating protocol is as follows:

count the inhibition zones,

cut out and identify these zones,

weigh each zone.

Each formulation is evaluated on a points scale (n=3). The averages of the different results are presented in graph form.

### Example 1

### Acrylic Patch of Dissolved Amorolfine HCl in the Absence of Absorption Promoter

Preparation of the Adhesive Matrix Layer

| | | % (m/m) |
|---|---|---|
| Constituents | % wt | Dry mass |
| Absolute ethanol | 58.43 | — |
| Ethyl acetate | 5.40 | — |
| Butyl acetate | 2.46 | — |
| Amorolfine HCl | 5.09 | 29.87 |
| Duro-Tak 367-2516 | 28.62 | 70.13 |

The preparation is limpid and fairly liquid. Acrylic polymer dissolves well in ethanol, as does amorolfine HCl.

The test results for the patches are as follows:

| Backing medium | BLF 2023 | BLF 2080 |
|---|---|---|
| Quantity deposited (g) | 2.42 | 2.95 |
| Adhesion relative to the | Detaches well | Detaches well |
| detachable outer | | |
| covering | | |
| Adhesion on nail | Adheres well | Adheres well |
| Thickness (μm) | 52 ± 19.4 | 96 ± 40.2 |

### Example 2

### Acrylic Patch of Dispersed Amorolfine HCl in the Absence of Absorption Promoter

The formulation of the adhesive matrix layer obtained is therefore as follows:

| | | % (m/m) |
|---|---|---|
| Constituents | % wt | Dry mass |
| Duro-Tak 387- | 84.73 | 69.98 |
| 2516 | | |
| amorolfine HCl | 15.27 | 30.02 |

The characteristics of the patch are as follows:

| Backing medium | BLF 2023 | BLF 2080 |
|---|---|---|
| Quantity deposited (g) | 2.99 | 5.50 |
| Adhesion relative to the | Detaches well | Detaches well |
| detachable outer covering | | |
| Adhesion on nail | Adheres well | Adheres well |
| Thickness (μm) | 203 ± 14.9 | 240 ± 11.9 |

The patches have a more opaque appearance as the amorolfine HCl crystals are very large.

The thicknesses are very large because the preparations are highly concentrated in amorolfine HCl and adhesive.

### Example 3

### Acrylic Patch of Dissolved Amorolfine HCl in the Presence of Urea/Lactic Acid Absorption Promoter

Preparation of the Adhesive Matrix Layer

| | | % (m/m) |
|---|---|---|
| Constituents | % wt | Dry mass |
| Absolute ethanol | 58.10 | — |
| Ethyl acetate | 5.28 | — |
| Butyl acetate | 2.21 | — |
| Lactic acid | 1.34 | 5.25 |
| Urea | 0.96 | 3.37 |
| amorolfine HCl | 5.20 | 28.74 |
| Duro-Tak 387- | 27.51 | 62.64 |
| 2516 | | |

The results of the tests carried out with the patches thus formulated are as follows:

| Backing medium | BLF 2023 | BLF 2080 |
|---|---|---|
| Quantity deposited (g) | 2.38 | 1.83 |
| Adhesion relative to the | Detaches well | Detaches well |
| detachable outer | | |
| covering | | |
| Adhesion on nail | Adheres well | Adheres well |
| Thickness (μm) | 86 ± 45.1 | 89 ± 38.1 |

According to the microscope images, with both backing layers, the amorolfine HCl is recrystallized.

Furthermore, with the backing medium BLF 2023, the adhesive detaches particularly well.

### Example 4

### Acrylic Patch of Dispersed Amorolfine HCl in the Presence of Urea/Lactic Acid Absorption Promoter

| | | % (m/m) |
|---|---|---|
| Constituents | % wt | Dry mass |
| Absolute | 9.55 | — |
| ethanol | | |
| Ethyl acetate | 51.20 | — |
| Butyl acetate | 2.01 | — |
| Lactic acid | 0.79 | 4.24 |
| Urea | 0.85 | 3.94 |
| Amorolfine HCl | 5.92 | 29.55 |
| Duro-Tak 387- | 29.68 | 62.27 |
| 2516 | | |

The results of the tests carried out with the patches thus formulated are as follows:

| Backing medium | BLF 2023 | BLF 2080 |
|---|---|---|
| Quantity deposited | 2.41 | 2.21 |
| (g) | | |
| Adhesion relative to | Detaches well | Detaches well |
| the detachable | | |
| outer covering | | |
| Adhesion on nail | Adheres well | Adheres well |
| Thickness (μm) | 60 ± 34.2 | 100 ± 48.2 |

The patches have good adhesive properties irrespective of the type of backing medium used.

### Example 5

### Acrylic Patch of Dissolved Amorolfine HCl in the Presence of Exaltolide Absorption Promoter

The formulation of the adhesive matrix layer obtained is as follows:

| | | % (m/m) |
|---|---|---|
| Constituents | % wt | Dry mass |
| Absolute ethanol | 56.78 | — |
| Ethyl acetate | 5.65 | — |
| Butyl acetate | 2.12 | — |
| Exaltolide | 2.99 | 14.93 |
| Amorolfine HCl | 5.84 | 29.23 |
| Duro-Tak 387- | 26.62 | 55.84 |
| 2516 | | |

The characteristics of the patches thus formulated are as follows:

| Backing medium | BLF 2023 | BLF 2080 |
|---|---|---|
| Quantity deposited | 2.53 | 1.32 |
| (g) | | |
| Adhesion relative to | Detaches well | Detaches well |
| the detachable | | |
| outer covering | | |
| Adhesion on nail | Adheres well | Adheres but |
| | | considerable |
| | | pressure required |
| Thickness (μm) | 86 ± 52.0 | 100 ± 61.4 |

The exaltolide recrystallizes irrespective of the backing medium.

More uniform recrystallization is observed with backing medium BLF 2080.

With this formulation, better adhesive properties are observed for backing medium BLF 2023.

### Example 6

### Acrylic Patch of Dispersed Amorolfine HCl in the Presence of Exaltolide

The formulation of the adhesive matrix layer obtained is as follows:

| | | % (m/m) |
|---|---|---|
| Constituents | % wt | Dry mass |
| Ethyl acetate | 25.83 | — |
| Butyl acetate | 0.87 | — |
| Exaltolide | 6.21 | 12.40 |
| amorolfine HCl | 12.5 | 25.04 |
| Duro-Tak 387-2516 | 54.59 | 62.56 |

The characteristics of the patches are as follows:

| Backing medium | BLF 2023 | BLF 2080 |
|---|---|---|
| Quantity deposited (g) | 2.46 | 2.87 |
| Appearance of | Backing uniform | Backing uniform |
| patches | opalescent | opalescent |
| Adhesion relative to | Detaches well | Detaches well |
| the detachable outer | | |
| covering | | |
| Adhesion on nail | Adheres well | Adheres well but |
| | | requires slight |
| | | pressure |
| Thickness (μm) | 223 ± 32.4 | 194 ± 47.1 |

The thicknesses are not significantly different between the two backing media.

The backing is uniform and good adhesive properties were obtained.

### Example 7

### Acrylic Patch of Dissolved Amorolfine in the Presence of N-acetyl-L-cysteine and Urea

The formulation of the adhesive matrix layer obtained is as follows:

| | | % (m/m) |
|---|---|---|
| Constituents | % wt | Dry mass |
| Absolute ethanol | 48.69 | — |
| Ethyl acetate | 4.32 | — |
| Butyl acetate | 2.22 | — |
| Urea | 2.49 | 9.83 |
| N-acetyl cysteine | 1.25 | 4.93 |
| amorolfine HCl | 7.58 | 29.82 |
| Duro-Tak 387- | 33.47 | 55.42 |
| 2516 | | |

The characteristics of the patches thus formulated are as follows:

| Backing medium | BLF 2023 | BLF 2080 |
|---|---|---|
| Quantity deposited | 5.24 g | 3.44 g |
| Adhesion relative to the | Detaches well | Detaches well |
| detachable outer | | |
| covering | | |
| Adhesion on nail | Adheres well | Adheres well |
| Thickness (μm) | 148 ± 41.8 | 105 ± 31.0 |

With the backing medium BLF 2023, recrystallization of the amorolfine HCl is more uniform.

The thickness is not identical for both backing media. The deposit on backing medium BLF 2023 is significantly thicker than that on backing medium BLF 2080.

Finally, it will be noted that backing medium BLF 2023 provides better detachment of the adhesive.

### Example 8

### Silicone Patch of Dispersed Amorolfine HCl in the Absence of Absorption Promoter

The formulations obtained are as follows:

| | | % (m/m) |
|---|---|---|
| Constituents | % wt | Dry mass |
| BIO PSA 7- | 78.42 | 70.23 |
| 4302 | | |
| amorolfine | 21.58 | 29.77 |
| HCl | | |

The characteristics of the patches are as follows:

| Backing medium | BLF 2023 | BLF 2080 |
|---|---|---|
| Quantity deposited | 2.33 | 2.71 |
| (g) | | |
| Adhesion relative to | Detaches | Detaches well |
| the detachable | well | |
| outer covering | | |
| Adhesion on nail | Adheres | Adheres well |
| | well | |
| Thickness (μm) | 421 ± 35.3 | 287 ± 35.7 |

The patches are very thick as there is little or no evaporation.

### Example 9

### Silicone Patch of Dispersed Amorolfine HCl in the Presence Urea and Lactic Acid

The following formulation is obtained:

| | | % (m/m) |
|---|---|---|
| Constituents | % wt | Dry mass |
| Absolute ethanol | 5.70 | — |
| Ethyl acetate | 67.58 | — |
| Butyl acetate | 1.91 | — |
| Urea | 0.48 | 2.59 |
| Lactic acid | 0.74 | 4.02 |
| amorolfine HCl | 5.38 | 29.15 |
| BIO PSA 7-4302 | 18.21 | 64.24 |

Absolute ethanol is necessary to dissolve the urea.

The patches have the following characteristics:

| Backing medium | BLF 2023 | BLF 2080 |
|---|---|---|
| Quantity deposited (g) | 3.53 | 3.74 |
| Appearance of | Bedding very uniform, | Bedding very |
| patches | very pale. Appears very | uniform, very pale. |
| | thin. | Appears very thin. |
| Adhesion on nail | Adheres well | Adheres well |
| Thickness (μm) | 33 ± 13.3 | 40 ± 24.1 |

The patches are very thin yet very homogeneous.

### Example 10

### Silicone Patch of Dispersed Amorolfine HCl in the Presence of Exaltolide

The following formulation is obtained:

| | | % (m/m) |
|---|---|---|
| Constituents | % wt | Dry mass |
| Ethyl acetate | 31.64 | — |
| Butyl acetate | 1.15 | — |
| Exaltolide | 7.66 | 14.79 |
| amorolfine HCl | 15.84 | 30.50 |
| BIO PSA 7-4302 | 43.71 | 54.71 |

Exaltolide is highly soluble in ethyl acetate. A supplementary solvent is not necessary. The final exaltolide content in the patch is 15% (m/m), with 30% amorolfine HCl.

The characteristics of the patches are as follows:

| | | |
|---|---|---|
| Backing medium | BLF 2023 | BLF 2080 |
| Quantity deposited (g) | 1.67 | 3.74 |
| Adhesion relative to the | Detaches poorly | Detaches poorly |
| detachable outer covering | | |
| Adhesion on nail | Adheres well | Adheres well |
| Detachment of nail | Fairly good | Fairly good |

The coating is uniform.

### Example 11

### Microbiological Assessment of the Patches Described in Examples 1 to 10

The operating conditions were identical for all patches, 72 hours of diffusion in the refrigerator and test at three different points with the hooves left on the agar during oven treatment.

Table of results:

| | Weight of inhibition | |
|---|---|---|
| | zones | |
| | (average of n = 3) | |
| Example | in mg | Standard deviation |
| Ex 1 | 80 | 23.26 |
| Ex 2 | 106.33 | 15.53 |
| Ex 3 | 192.67 | 2.31 |
| Ex 4 | 186.67 | 49.01 |
| Ex 5 | 81.67 | 7.77 |
| Ex 6 | 193.33 | 51.55 |
| Ex 7 | 167.33 | 39.5 |
| Ex 8 | 86.67 | 23.8 |
| Ex 9 | 202.33 | 91.6 |
| Ex 10 | 128.33 | 27.47 |

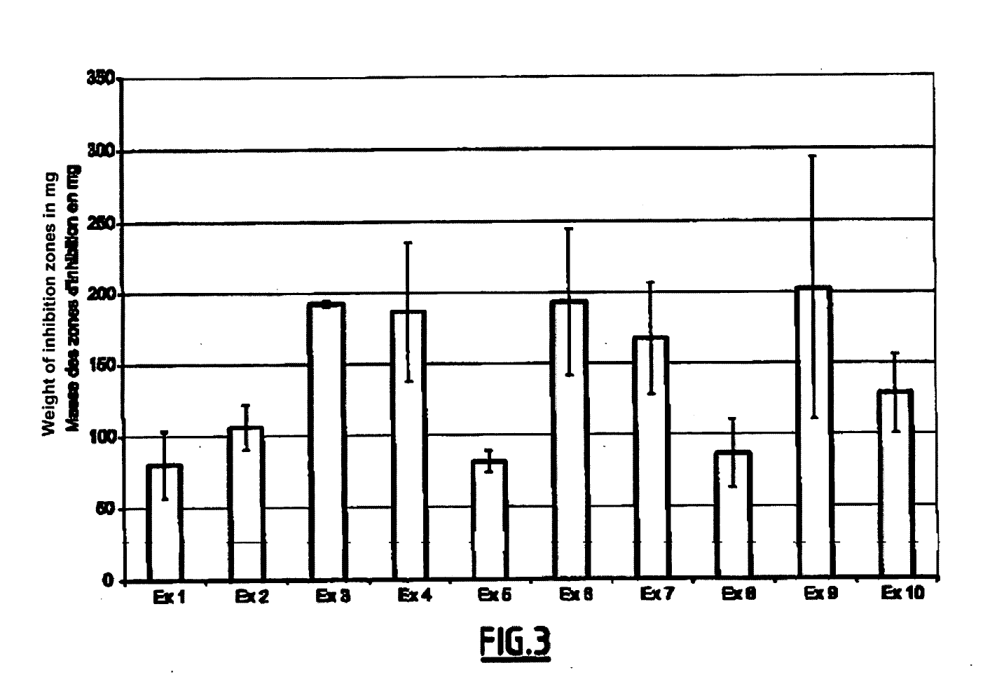

FIG. 3 summarizes the results of all the tests carried out on the patches.

This microbiological assessment demonstrates the ability of amorolfine HCl to diffuse from patches of different formulations. This diffusion does not appear to be dependent on the nature of the adhesive (acrylic or silicone), nor dependent on the preparation of the pre-dissolved or dispersed active agent.

However, it does show the effect of the absorption promoters on the diffusion of amorolfine HCl from the patches.

Thus, the performance results are generally consistent for urea and lactic acid with an increase in standard effectiveness of the order of 114% compared with patches without absorption promoters.

In the case of exaltolide, the increases in effectiveness are highly variable, ranging from 2.5% for amorolfine HCl dissolved with Duro-Tak® 387-2516 to 121% for amorolfine HCl dispersed in BIO PSA 7-4302®. Exaltolide therefore appears to be slightly less effective as an absorption promoter, with variable results depending on the nature of the adhesive.

For the urea/N-acetyl-L-cysteine combination, the increase relative to the corresponding formulation without promoter is 108%. N-acetyl-L-cysteine therefore provides an alternative to lactic acid when it is combined with urea.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A transungual patch/device useful for the treatment of onychomycosis, comprising:
(i) a backing medium; and
(ii) an adhesive matrix layer deposited thereon which comprises (a) a thus effective amount of amorolfine active agent or derivative thereof essentially uniformly distributed therethrough, dissolved or dispersed in said adhesive, and (b) at least one absorption promoter for enhancing the absorption of said amorolfine or derivative into the nail onto which said patch/device is applied, said at least one absorption promoter comprising either exaltolide alone, or at least two such absorption promoters selected from the group consisting of urea, N-acetylcysteine and lactic acid.

2. The transungual patch/device as defined by claim 1, said adhesive matrix layer comprising a pharmaceutically acceptable salt of amorolfine.

3. The transungual patch/device as defined by claim 2, said adhesive matrix layer comprising amorolfine hydrochloride.

4. The transungual patch/device as defined by claim 1, in anhydrous state.

5. The transungual patch/device as defined by claim 1, said adhesive matrix layer being devoid of additives.

6. The transungual patch/device as defined by claim 1, said at least one absorption promoter comprising urea and lactic acid, or urea and N-acetylcysteine.

7. The transungual patch/device as defined by claim 1, said at least one absorption promoter comprising exaltolide alone.

8. The transungual patch/device as defined by claim 1, said amorolfine active agent or derivative thereof comprising from 2% to 70% by weight of said adhesive matrix layer.

9. The transungual patch/device as defined by claim 1, said amorolfine active agent or derivative thereof comprising from 20% to 40% by weight of said adhesive matrix layer.

10. The transungual patch/device as defined by claim 1, said adhesive matrix layer comprising a pressure-sensitive adhesive.

11. The transungual patch/device as defined by claim 1, said adhesive matrix layer comprising a polyacrylate, polyisobutylene or silicone adhesive, or mixture thereof.

12. The transungual patch/device as defined by claim 1, said adhesive comprising from 10% to 98% by weight of said adhesive matrix layer.

13. The transungual patch/device as defined by claim 1, said adhesive comprising from 25% to 85% by weight of said adhesive matrix layer.

14. The transungual patch/device as defined by claim 1, said backing medium comprising a polyurethane, poly(vinyl ethylene-co-acetate), polyethylene or polyvinyl chloride polymer, or mixture thereof.

15. The transungual patch/device as defined by claim 1, each such absorption promoter comprising from 1 % to 30% by weight of said adhesive matrix layer.

16. The transungual patch/device as defined by claim 1, each such absorption promoter comprising from 2.5% to 15% by weight of said adhesive matrix layer.

17. The transungual patch/device as defined by claim 6, said adhesive matrix layer comprising 2% to 6% by weight of each of urea and lactic acid.

18. The transungual patch/device as defined by claim 6, said adhesive matrix layer comprising from 2% to 10% by weight of each of urea and N-acetylcysteine.

19. The transungual patch/device as defined by claim 7, said adhesive matrix layer comprising from 10% to 20% by weight of exaltolide.

20. The transungual patch/device as defined by claim 1, further comprising a detachable outer covering deposited onto the face surface of said adhesive matrix layer.

21. A transungual patch/device useful for the treatment of onychomycosis, consisting essentially of:
(i) a flexible backing medium of a size and shape to be adhered only onto a nail surface; and
(ii) an adhesive matrix layer deposited thereon which comprises (a) a thus effective amount of amorolfine active agent or derivative thereof essentially uniformly distributed therethrough, dissolved or dispersed in said adhesive, and (b) at least one absorption promoter for enhancing the absorption of said amorolfine or derivative into the nail onto which said patch/device is applied, said at least one absorption promoter comprising either exaltolide alone, or at least two such absorption promoters selected from the group consisting of urea, N-acetylcysteine and lactic acid.

22. A transungual patch/device useful for the treatment of onychomycosis, comprising:
(i) a backing medium;
(ii) at least two matrix layers deposited thereon, each of which comprises a thus effective amount of amorolfine active agent or derivative thereof, at least one of said matrix layers comprising an adhesive matrix layer, and at least one of said matrix layers comprising at least one absorption promoter for enhancing the absorption of said amorolfine or derivative into the nail onto which said patch/device is applied, said at least one absorption promoter comprising either exaltolide alone, or at least two such absorption promoters selected from the group consisting of urea, N-acetylcysteine and lactic acid; and
(iii) a membrane between said at least two matrix layers permitting gradual release of said amorolfine or derivative thereof into the nail.

23. The transungual patch/device as defined by claim 22, further comprising a detachable outer covering deposited onto the outermost face surface of said at least two matrix layers.

24. A method for fabricating a transungual patch/device as defined by claim 1, comprising:
(i) preparing a mixture of (1) adhesive, (2) either exaltolide or at least two absorption promoters selected from the group consisting of urea, N-acetylcysteine and lactic acid and (3) amorolfine or derivative thereof, in a solvent, said adhesive being miscible with said solvent;
(ii) depositing the mixture thus obtained onto a backing medium; and
(iii) evaporating the solvent therefrom.

25. The method as defined by claim 24, said adhesive comprising a polyacrylate.

26. The method as defined by claim 24, said adhesive comprising a silicone.

27. The method as defined by claim 24, said solvent comprising ethanol.

28. The method as defined by claim 24, said solvent comprising dichloromethane.

29. The method as defined by claim 24, said amorolfine or derivative thereof being dissolved in said mixture obtained at (i).

30. The method as defined by claim 24, said amorolfine or derivative thereof being dispersed in said mixture obtained at (i).

31. The method as defined by claim 24, said mixture obtained at (i) further comprising a co-solvent capable of dissolving said amorolfine or derivative thereof and/or said absorption promoter(s).

32. The method as defined by claim 31, said co-solvent comprising isopropanol.

33. The method as defined by claim 31, said solvent and co-solvent constituting 2% to 90% by weight of the mixture obtained at (i).

34. The method as defined by claim 24, further comprising (iv) placing a detachable outer covering onto the face surface of said deposited adhesive layer.

35. A regime or regimen for the treatment of onychomycosis, comprising adhering the transungual patch/device as defined by claim 1 onto the affected nail surface(s) of an individual in need of such treatment and maintaining said patch/device in place until eliciting the desired response.
